(19) United States
(12) Patent Application Publication
(10) Pub. No.: US 2026/0115444 A1
(43) Pub. Date: Apr. 30, 2026

(54) Dual enteral feed connector

(71) Applicant: Linda Yeow, Yonkers, NY (US)

(72) Inventor: Linda Yeow, Yonkers, NY (US)

(21) Appl. No.: 18/928,184

(22) Filed: Oct. 28, 2024

(51) Int. Cl. A61M 39/10

(52) U.S. Cl. CPC A61M 39/10

(57) ABSTRACT

This invention enables the healthcare provider to provide enteral nutrition and flush medication through the Dual enteral feed Connector® efficiently. This invention will permit the flow of nutrition and administer medication through the infusion ports. This tool will help to promote and complete task efficiently for healthcare provider at feeding tube site.

### BACKGROUND OF THE INVENTION

The purpose of this invention helps to administer medication and provide enteral nutrition for patients with feeding tubes. The tool serves as a good purpose to make tasks performance efficient at feeding tube site.

### SUMMARY

This invention will help patients to receive enteral nutrition and medication efficiently on the tool itself attached on feeding tube site. The healthcare provider will be able to flush medication properly and provide enteral nutrition through the ports of the tool. In addition, this tool will promote proper care for patients with feeding tubes.

### BRIEF DESCRIPTION OF THE DRAWINGS

In this invention, I will share the drawing and what are the components on it. The tool in the drawing will function as a way to provide enteral nutrition and flush medication through these infusion ports.

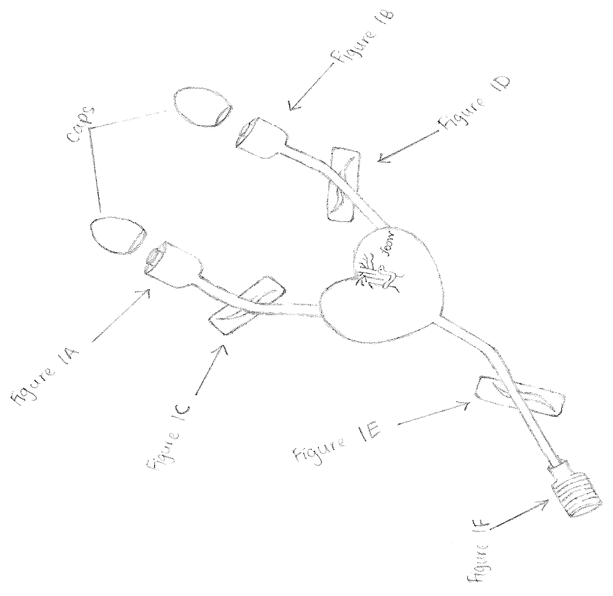

In FIG. 1 drawing, the drawing shows a tool that will attach itself to a feeding tube site. The tool will function as a useful resource to provide enteral nutrition and administer medication to feeding tube site. I named this tool as Dual enteral feed Connector®.

The tool serves as a useful resource to perform tasks for patients with feeding tubes. The tool consists of two infusion ports, three clamps, two caps and a connector. In the drawing, two infusion ports are identified as FIG. 1A and FIG. 1B. Clamps on the tool are identified as FIG. 1C, FIG. 1D and FIG. 1E. The connector identified as FIG. 1F is found at end of the tool; it will attach onto a feeding tube for securement. FIG. 1G shows fastening caps that will screw onto the infusion ports when not in used.

### DETAILED DESCRIPTION

I will show you how the invention tool works in this section. Using the Dual enteral feed Connector®, you will need to first attach the end of the connector (FIG. 1F) into a feeding tube site. After attaching the connector, you will clamp all the three clamps (FIG. 1C, FIG. 1D and FIG. 1E) on the tool. If the infusion ports are in use, all three clamps should be unclamped. You can begin to give enteral nutrition to first infusion port (FIG. 1A) and administer medication through the second infusion port (FIG. 1B) by using a feeding syringe. If the ports are not in use, clamping of the clamps will prevent fluid backflow or leakage out of the ports. Finally, fastening caps (FIG. 1G) can be used to cover the ports when not in used.

The invention claimed is:

1. A Dual enteral feed Connector® comprising;
a first infusion port;
a second infusion port;
a connector;
wherein the first infusion port is connected by a flex catheter line, the second infusion port is connected by a second flex catheter line, and a connector is connected by a third flex catheter line;
a first clamp disposed on the first flex catheter line;
a second clamp disposed on the second flex catheter line;
a third clamp disposed on the third flex catheter line;
a connector with dual infusion ports,
wherein both infusion ports are configured to attach to securement caps.
